(19) United States
(12) Patent Application Publication
(10) Pub. No.: US 2010/0041918 A1
(43) Pub. Date: Feb. 18, 2010

(54) CYCLOPENTENE DIOL MONOACETATE DERIVATIVES

(75) Inventor: Kurt Laumen, March (DE)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG

(21) Appl. No.: 12/312,311

(22) PCT Filed: Nov. 5, 2007
(86) PCT No.: PCT/EP2007/061886

§ 371 Date: May 4, 2009

(30) Foreign Application Priority Data
Nov. 10, 2006 (EP) 06123845.7

(51) Int. Cl. C07C 61/06

(52) U.S. Cl. 562/503

(57) ABSTRACT

A process for the preparation of organic compounds of formula (I), wherein R¹ is as described herein.

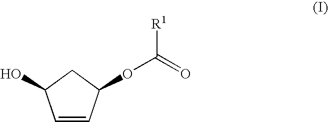

This invention relates to a preparation of organic compounds, particularly a cyclopentene diol monoacetate derivative compound of formula (I):

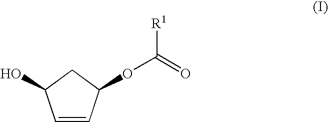

where R¹ is selected from the group consisting of C₁-C₈-alkyl, C₆-C₁₀-aryl, C₁-C₈-alkoxy and C₆-C₁₀-aryloxy.

Homochiral cyclopentene diol monoacetate derivatives a-d and diols e-f have been used as a key building block for the synthesis of a wide range of important molecules, and in particular prostanoids and carbocyclic nucleosides.

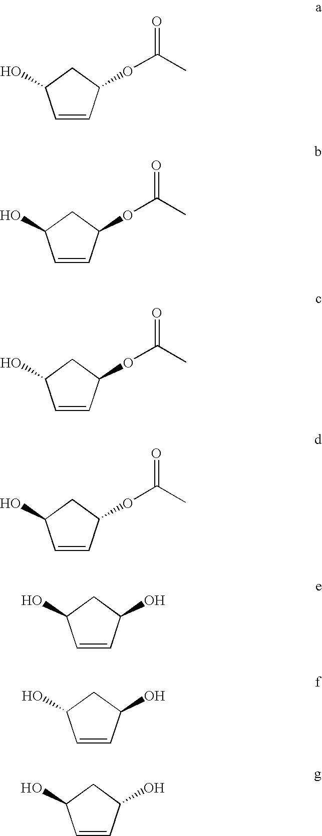

Current routes to the cis enantiomers a and b involve hazardous starting materials/intermediates (cyclopentadiene and peroxides) and operations and or capricious reactions, and or poor selectivity which limits their efficiency and utility, in particular, for scale up.

Cyclopentene diol monoacetates a and b have been prepared through singlet oxygen addition to cracked cyclopentadiene dimer followed by reduction of the peroxide. See Saito et al., “Structure-activity relationships of untenone A and its derivatives for inhibition of DNA polymerases” Frontier Research Center for Genome and Drug Discovery, Tokyo University of Science, Noda, Chiba, Japan, Bioorg Med Chem Lett, Vol. 14, No. 8, pp. 1975-1977 (2004); and Zhang et al., “Versatile Photosensitization System for 1O2-Mediated Oxidation of Alkenes Based on Nafion-Supported Platinum(ll) Terpyridyl Acetylide Complex”, Technical Institute of Physics and Chemistry, Chinese Academy of Sciences, Beijing, Peop. Rep. China, Org Lett, Vol. 5, No.18, pp. 3221-3224 (2003). The diol can be diacylated then enzymatically desymmetrised to provide 1 or 2. See Lalonde et al., “Cross-Linked Crystals of Candida rugosa Lipase: Highly Efficient Catalysts for the Resolution of Chiral Esters”, Altus Biologics Inc., Cambridge, Mass., USA, JACS, Vol. 117, No. 26, pp. 6845-6852 (1995).

Bromination of cyclopentadiene has also been described followed by acetate displacement, but suffers from low yields. See DePuy and Zaweski, “Cyclopentene-3,5-dione. I. Synthesis and properties”, Iowa State Univ., Ames, JACS, Vol. 81, pp. 4920-4924 (1959).

Peracid oxidation of cyclopentadiene has also been used to prepare the diol precursors to 1-4 but suffers from poor regio and stereo selectivity. See Reimann and Poeschl, “Intramolecular alkylation of aromatic compounds. Part 32. Regioselective synthesis of 4-methyl-1-pyrindan-5-one”, Inst. Pharm. Lebensmittelchemie, Univ. Muenchen, Munich, Germany, Pharmazie, Vol. 50, No. 9, pp. 589-592 (1995).

The diol for the trans-isomers f and g have been prepared chiraly by a long synthetic sequence. See Kimura, Ehama and Inomata, “Chiral preparation of C2-symmetric 4-cyclopentene-1,3-diol”, Tohoku Pharmaceutical University, Sendai, Japan, Synthesis, pp. 1027-1032 (2002).

A more efficient method for producing homochiral cyclopentene diol monoacetate derivatives is therefore desirable. Such a method would provide high purity compounds and be suitable for large scale synthesis.

The present invention relates to the preparation of organic compounds of formula (I):

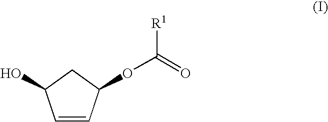

where R¹ is selected from the group consisting of C₁-C₈-alkyl, C₆-C₁₀-aryl, C₁-C₈-alkoxy and C₆-C₁₀-aryloxy, comprising the steps of:
(1) reacting a furfuryl alcohol in an acidic solution for a time sufficient to form a compound of formula (II):

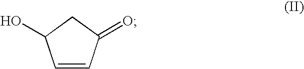

(2) reacting a compound of formula (II) with a protecting group in an aprotic solvent in the presence of base for a time sufficient to form a compound of formula (III):

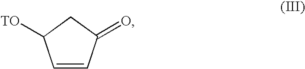

where T is a protecting group;
(3) reducing a compound of formula (III) and removing said protecting group of said compound of formula (III) to provide a compound of formula (IV):

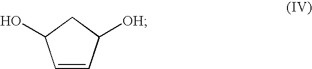

(4) reacting a compound of formula (V):

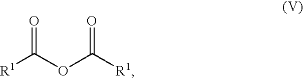

where each R¹ is independently selected from C₁-C₈-alkyl, C₆-C₁₀-aryl, C₁-C₈-alkoxy, and C₆-C₁₀-aryloxy, or
a compound of formula (Va):

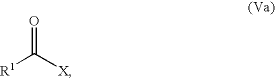

where X is selected from the group consisting of halogen, imidazole or N-hydroxybenzotriazole with a compound of formula (IV) to provide a compound of formula (VI):

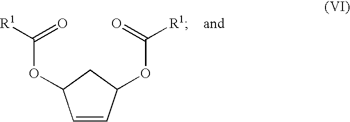

(5) reacting a compound of formula (VI) with an enzyme to provide a compound of formula (I).

### DEFINITIONS

The following terms and abbreviations are used herein and defined as follows.

“DMAP” is 4-dimethylaminopyridine.

“MTBE” is methyl t-butyl ether.

“DIBAL-H” is diisobutylaluminium hydride, or DIBAH, and is a reducing agent with the formula ⁱBU2AlH, where ⁱBu represents an isobutyl group.

The reactions of the synthetic methods claimed herein are carried out in suitable solvents which may be readily selected by one of skill in the art of organic synthesis, said suitable solvents generally being any solvent which is substantially non-reactive with the starting materials (reactants), the intermediates, or products at the temperatures at which the reactions are carried out, i.e., temperatures which may range from the solvent's freezing temperature to the solvent's boiling temperature. A given reaction may be carried out in one solvent or a mixture of more than one solvent. Depending on the particular reaction step, suitable solvents for a particular reaction step may be selected.

Suitable aprotic solvents may include, by way of example and without limitation, tetrahydrofuran, benzene, chlorobenzene, o-, m-, p-dichlorobenzene, dichloromethane, toluene, hexane, cyclohexane, pentane, methyl t-butyl ether, N-methylpyrrolidine, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene or hexamethylphosphoramide.

As used herein, the term “base” refers to any base known to those skilled in the art that are basic enough to deprotonate an alcohol in situ while still being compatible with carbonyls in situ, such as triethylamine, tributylamine, piperidine, pyrrolidine, pyridine, N,N-diisopropylethylamine and N,N-diisopropylamine.

“Halo” or “halogen”, as used herein, refers to fluoro, chloro and bromo.

“C₁-C₈-Alkyl”, as used herein, is intended to include both branched and straight chain saturated aliphatic hyrodocarbon groups.

“C₆-C₁₀-Aryl”, as used herein, is intended to include an aromatic carbocyclic group that contains 6-10 carbon atoms and which may be, e.g., a monocyclic group, such as phenyl; or a bicyclic group, such as naphthyl.

“C₁-C₈-Alkoxy”, as used herein, denotes straight chain or branched alkoxy having 1-8 carbon atoms, e.g., O—C₁-C₈-alkyl.

“C₆-C₁₀-Aryloxy”, as used herein, denotes an aryl as herein defined linked to an oxygen, e.g. O-aryl.

The enzyme, which is used in the present invention, is not particularly limited to but includes lipase, esterase, acylase, and so on.

Preferable are a lipase derived from microorganisms which belong to Alkaligenes, a lipase derived from microorganisms which belong to Candida, a lipase derived from microorganisms which belong to Pseudomonas, a lipase derived from microorganisms which belong to Mucor, and the like.

The above lipase derived from microorganisms which belong to Alkaligenes includes “Lipase PL” (a registered trademark of product of MEITO SANGYO Co.) and so on. The above lipase derived from microorganisms which belong to Candida includes “Novozym 435” (also referred to as “Novo SP435”) (registered trademarks of product of Novo-Nordisk A/S), “Lipase OF” (a registered trademark of product of MEITO SANGYO Co.), “Lipase MY” (a registered trademark of product of MEITO SANGYO Co.) and so on. The above lipase derived from microorganisms which belong to Pseudomonas includes “Lipase PS AMANO” (a registered trademark of product of AMANO PHARMACEUTICAL Co.) and so on. The above lipase derived from microorganisms which belong to Mucor includes “Lipozyme IM” (a registered trademark of product of Novo-Nordisk A/S).

The compounds herein described may have asymmetric centers. All chiral, diastereomeric, and racemic forms are included in the present invention. It will be appreciated that certain compounds of the present invention contain an asymmetrically substituted carbon atom, and may be isolated in optically active or racemic forms. It is well known in the art how to prepare optically active forms, such as by resolution of racemic forms or by synthesis, from optically active starting materials. All chiral, diastereomeric, racemic forms and all geometric isomeric forms of a structure are intended, unless the specific stereochemistry or isomer form is specifically indicated.

Combinations of substituents and/or variables are permissible only if such combinations result in stable compounds. By stable compound or stable structure it is meant herein a compound that is sufficiently robust to survive isolation to a useful degree of purity from a reaction mixture.

The present invention is contemplated to be practiced on at least a multigram scale, kilogram scale, multikilogram scale or industrial scale. “Multigram scale”, as used herein, is preferably the scale wherein at least one starting material is present in 10 grams or more, more preferably at least 50 grams or more, even more preferably at least 100 grams or more. “Multikilogram scale”, as used herein, is intended to mean the scale wherein more than 1 kilogram of at least one starting material is used. “Industrial scale”, as used herein, is intended to mean a scale which is other than a laboratory scale and which is sufficient to supply product sufficient for either clinical tests or distribution to consumers.

The protecting group T may be chosen from suitable protecting groups for the nature of the functional group, e.g., as described in Protective Groups in Organic Synthesis, T. W. Greene and P. G. M. Wuts, John Wiley & Sons Inc, Second Edition (1991), which reference also describes procedures suitable for replacement of the protecting groups by hydrogen.

Reducing or the reduction step(s) are carried out using known procedures for reducing ketones or analogously e.g. as hereinafter described in the Examples.

“Nucleophilic catalyst” catalyzes a variety of reactions. An example of a nucleophilic catalyst includes, but is not limited to, DMAP. Examples of reactions includes esterifications with anhydrides, Baylis-Hillman reaction, silylabon, tritylation, Steglich-Rearrangement, Staudinger synthesis of β-lactams and many more as described in Berry et al., “Catalysis by 4-dialkylaminopyridines” and Höfle, Steglich and Vorbrüggen, “O-401R 2001 and 4-Dialkylaminopyridines as Highly Active Acylation Catalysts”, Angew Chem Int Ed Engl, Vol. 17, pp. 569-583 (1978).

According to the preparation of a compound of formula (I), the protecting group in step (2) is suitably chloro-trimethylsilane.

According to the preparation of a compound of formula (I), the compound of formula (V) is preferably

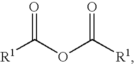

where each R¹ is independently suitably C₁-C₈-alkyl. More preferably, the compound of formula (V) is acetic anhydride.

According to the preparation of a compound of formula (I), the enzyme in step (5) is suitably Novo SP435 or Lipase PS Amano.

Another aspect of the invention provides for the preparation of organic compounds of formula (Ia):

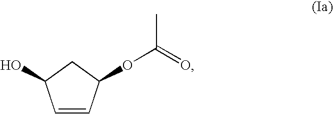

comprising the steps of:
(1) reacting a furfuryl alcohol in an acidic solution comprising water for a time sufficient to form a compound of formula (IIa):

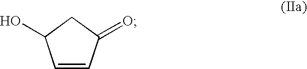

(2) reacting a compound of formula (IIa) with chloro-trimethylsilane in dichloromethane in the presence of base for a time sufficient to form compound of formula (IIIa):

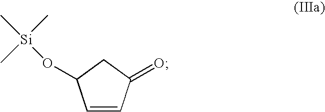

(3) reducing a compound of formula (IIIa) in an aprotic solvent to provide racemic mixture of a compound of formula (IVa):

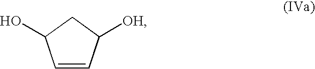

(4) reacting said racemic mixture of a compound of formula (IVa) with acetic anhydride in an aprotic solvent in the presence of base for a time sufficient to form a compound of formula (VIa):

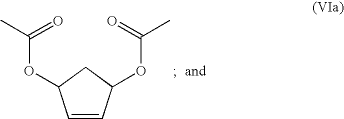

(5) reacting a compound of formula (VIa) with Novo SP435 or Lipase PS Amano (LPS AB0351302) to provide a compound of formula (Ia).

According to the preparation of the compound of formula (Ia), the acidic solution of step (1) comprises potassium hydrogen phosphate and ortho phosphoric acid.

According to the preparation of the compound of formula (Ia), the acidic solution of step (1) has a pH of about 3.0 to about 5.0.

According to the preparation of the compound of formula (Ia), the base of step (2) is suitably triethylamine.

According to the preparation of the compound of formula (Ia), step (2) further comprises a nucleophilic catalyst, such as DMAP.

According to the preparation of the compound of formula (Ia), DIBAL-H, is used as a reducing agent in step (3).

According to the prepartion of the compound of formula (Ia), the aprotic solvent of step (3) is suitably toluene or tert-butyl methyl ether. Preferably, the aprotic solvent is a mixture of toluene and tert-butyl methyl ether.

According to the preparation of the compound of formula (Ia), the base of step (4) is suitably triethylamine.

According to the preparation of the compound of formula (Ia), step (4) further comprises a nucleophilic catalyst, such as DMAP.

According to the preparation of the compound of formula (Ia), the aprotic solvent in step (4) is suitably dichloromethane.

According to the preparation of the compound of formula (Ia), step (5) provides an enantiomeric ratio of the product, compound (Ia), of at least 80%. Preferably, the enantiomeric ratio of the product, compound (Ia), is at least 90%.

It is to be understood that one skilled in the art of organic synthesis could prepare the methods described or exemplified herein to prepare homologues of compounds of formulae (I)-(V) and/or compounds of formulae (Ia)-(Va).

Scheme 1 outlines the key steps in the synthesis of cyclopentene diol monoacetate derivative, such as acetic acid (1S,4R)-4-hydroxy-cyclopent-2-enyl ester 6.

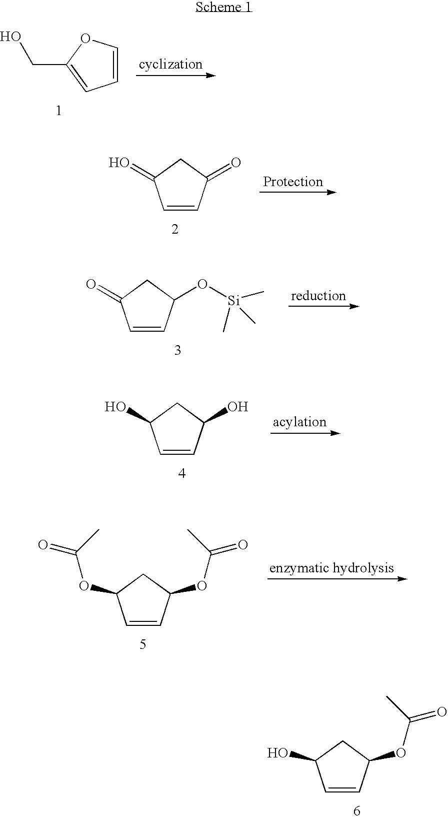

The process of the present invention describes the generation of a more efficient method for producing homochiral cyclopentene diol monoacetate derivatives in high purity. The process also, does not involve hazardous starting materials/intermediates (cyclopentadiene and peroxides) and operations and or capricious reactions, and or poor selectivity which limits their efficiency and utility for scale up.

Scheme 2 describes the process of preparing 4-hydroxy-cyclopen-2-enone 2.

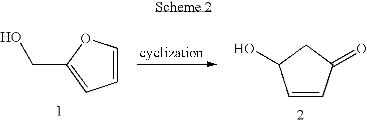

In scheme 2, an acid, preferably orthophosphoric acid, is added to a solution of furfuryl alcohol and potassium hydrogen phosphate in water, adjusting the pH solution to about 4.1. Afterwards, the solution is heated at reflux for a sufficient period of time to generate the 4-hydroxy-cyclopen-2-enone 2.

In scheme 3, the 4-hydroxy-cyclopen-2-enone 2 is protected with a protecting group, such as chloro-trimethylsilane. In the protection process, base is added to a solution of an aprotic solvent, such as dichloromethane followed by DMAP. This resultant solution is cooled to about 0° C. and chloro-trimethylsilane is added while maintaining the temperature below 10° C. The reaction is stirred for a sufficient time to generate 4-trimethylsilanoxy-cyclopent-2-enone 3.

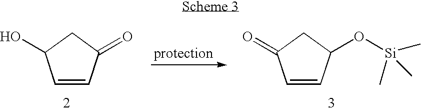

In scheme 4, the carbonyl group is reduced and the protecting group is removed to provide a racemic mixture of 4-cyclopenten-1,3-diol 4. DIBAL-H is added to a solution of 4-trimethylsilaoxy-cyclopent-2-enone 3, in an aprotic solvent under an inert atmosphere, such as nitrogen or argon, at a temperature below 0° C. Preferably between −20° C. and −30° C. The resultant reaction mixture is stirred for a time sufficient to generate a racemic mixture of 4-cyclopenten-1,3-diol 4.

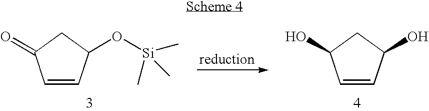

Scheme 5 provides a process of generating acetic acid (1S,4R)-acetoxy-cyclopent-2-enyl ester 5, in good yield.

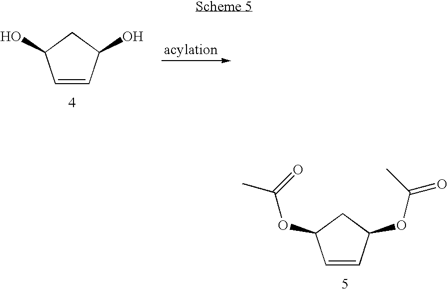

At about room temperature, a suspension of 4-cyclopenten-diol 4, in an aprotic solvent, such as dichloromethane, base is added, such as triethylamine, followed by DMAP. An anhydride or acyl halide, preferably acetic anhydride is added to the resultant mixture at a temperature below 25° C., usually in the range from 0-20° C. After this addition the resultant reaction mixture is warmed to about room temperature for a sufficient time to generate acetic acid (1S,4R)-acetoxy-cyclopent-2-enyl ester 5.

In scheme 6, hydrolysis of the diacetate 5, to the monoacetate 6, while providing good yield is accomplished by adding (1S,4R)-acetoxy-cyclopent-2-enyl ester 5 to a phosphate buffer, such as pH 7 (Fluka 73173), an enzyme, such as Lipase PS Amano and adjusting the pH, if necessary, to about 7 with base for a time sufficient to generate compound 6. The base is preferably 1 M NaOH.

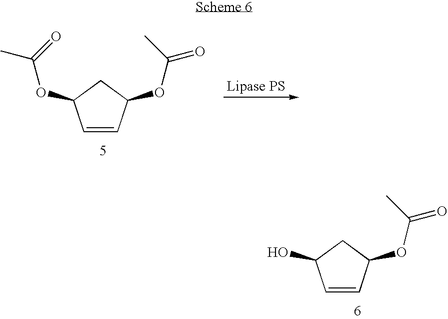

Alternatively, the C2-symmetric trans alcohols c and d could also be obtained by a variant of the routes described herewith with a resolution (enzymatic) followed by a trans selective reduction of the alcohol directly:

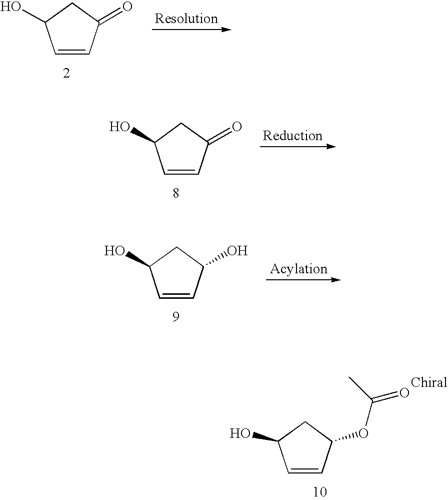

The following examples are meant to be illustrative of the present invention. These examples are presented to exemplify the invention and are not to be construed as limiting the invention's scope.

### Preparation of 4-Hydroxy-cyclopen-2-enone 2

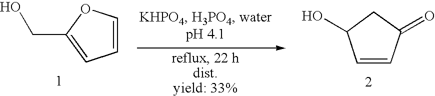

To a stirred solution of furfurylalcohol 1 (1.2 L, 13.85 mol) in water (24 L), potassium hydrogen phosphate (69 g, 0.507 mol) is added. Then 2 mL of ortho phosphoric acid is added dropwise to adjust the pH from 4.5 to 4.1. Afterwards, the solution is heated to 99° C. and stirred overnight at this temperature, while the clear yellow solution turns into a light brown gummy suspension. The pH decreases to 4.0. The reaction mixture is cooled to 60° C. and filtered through hyflo. The clear, yellow filtrate is concentrated under reduced pressure (70° C., 10 mbar,) and the resulting brown solid is suspended in dichloromethane (4 L) and stirred for 15 minutes. The suspension is then separated by filtration and the mother liquor is evaporated to dryness. The crude product (606 g) is purified by distillation at 130° C., 0.0045 mbar over a short path distillation column to give 4-Hydroxy-cyclopent-2-enone 2 (448.8 g, 33%) as a colorless liquid.

### Preparation of 4-Trimethylsilanoxy-cyclopent-2-enone 3

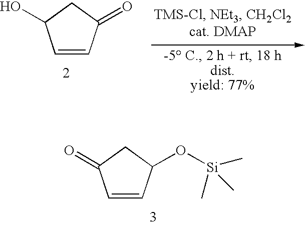

To a solution of 4-hydroxy-cyclopent-2-enone 2 (400 g, 4.01 mol) in 5 L of dichloromethane, triethylamine (781.5 mL, 5.614 mol) is added, followed by 4-DMAP (8 g, 0.064 mol). The mixture is cooled to 0° C. and chloro-trimethylsilane (560 mL, 4.42 mol) is added dropwise keeping the temperature between 0° C. and 5° C. The formed thick yellow suspension is diluted with THF (1 L) and then stirred at room temperature for about 1 hour. The suspension is diluted with dichloromethane (5 L) and extracted twice with 15% aqueous ammonium chloride (5 L each). The aqueous layers are extracted with dichloromethane (3 L). The combined organic layers are dried over Na₂SO₄, filtered and evaporated under vacuum to dryness. The crude product (672.8 g) is purified by short path distillation at 80° C., 0.009 mbar to afford 445.5 g of 4-trimethylsilanoxy-cyclopent-2-enone, 3 as a yellow oil (77%).

### Preparation of racemic 4-Cyclopenten-1.3-diol 4

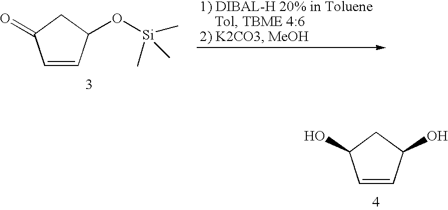

To a cooled (−30° C.) solution of 4-trimethylsilanoxy-cyclopent-2-enone 3 (520 g, 3.05 mol) in a mixture of 2.1 L of TBME and 3.1 L of toluene, DIBAL-H 20% in toluene (5.05 L, approx. 5.05 mol) is added dropwise under an argon atmosphere, keeping the temperature between −22° C. and −25° C. The reaction mixture is stirred at −22° C. for 1 hour, (at which time, TLC shows complete conversion of the starting material), then warmed to 0° C. and quenched carefully with saturated NH₄Cl (350 mL). The temperature is kept between 0° C. and 25° C. by cooling with a CO₂-EtOH bath. The mixture is diluted with MeOH (10 L), hyflo (125 g) is added and the mixture is stirred for 1 hour. The suspension is suction filtered. The filter cake is washed with MeOH (5 L) as described previously. The combined filtrates are evaporated to dryness under reduced pressure to afford crude 4-cyclopenten-1.3-diol 4 (329 g, 3.28 mol, quantitative) as a red amorphous solid. The crude product is used in the next step.

### Preparation of Acetic acid (1S,4R)-4-acetoxy-cyclopent-2-enyl ester 5

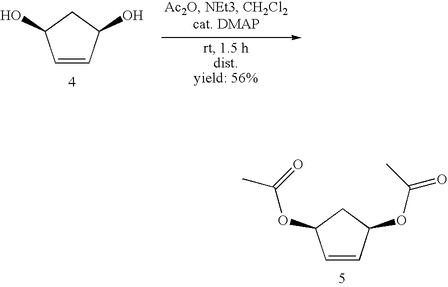

At room temperature, a suspension of 4-cyclopenten-diol 4 (329 g, 3.05 mol) in dichloromethane.(3.2 L) is treated with triethylamine (1.27 L, 9.15 mol), and 4-DMAP (11.2 g, 0.09 mol). Acetic anhydride is added dropwise, keeping the temperature between 80 and 19° C. by cooling with an ice-bath. The mixture is stirred for 2 hours at room temperature. TLC shows complete conversion of the starting material. The reaction mixture is poured into a well stirred solution of 2 M aqueous HCl (5 L). After 15 minutes well stirring, the water layer is separated and then extracted with dichloromethane (4 L). The combined organic layers are extracted sequentially with water (2×2.5 L) and with brine (2.5 L), then dried over Na₂SO₄, filtered and evaporated to dryness under reduced pressure to give 482 g of crude product as a brown oil. The product is purified by short path distillation at 60° C., 0.8 mbar to give acetic acid (1S,4R)-4-acetoxy-cyclopent-2-enyl ester 5 (370 g, 68% over 2 steps) as light yellow liquid. Chemical purity: 87% cis and 13% trans isomer, analyzed by GC-MS.

### Preparation of Acetic acid (1S,4R)-4-hydroxy-cyclopent-2-enyl ester 6

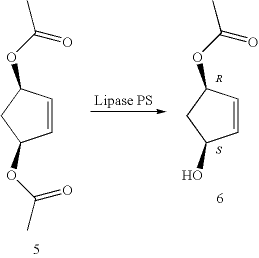

382.68 g of acetic acid (1S,4R)-4-acetoxy-cyclopent-2-enyl ester 5 is added to 2, 100 g phosphate buffer pH 7 and the pH is adjusted to pH 7 with 1 M NaOH. 4 g Lipase PS Amano (LPS AB0351302) is added to the reaction mixture and the reaction mixture stirred overnight.

The reaction mixture is transfered into the organic layer via extraction with DCM (checked by TLC of aqueous layer in DCM/MeOH 95:5). The organic layer is dried over MgSO₄, filtered and evaporated to 280 g yellow oil.

The oil is dissolved heated ether and precipitated with hexane. The white crystals are filtered and dried in the vacuum oven to provide 174.02 g→58.9% ee: >99.9% chemical purity: >99.0%; cis/trans ratio: >99.9/0.1; [α]_{D}²⁰=+64.4° (c=1; CHCl₃).

Unreacted acetic acid (1S,4R)-4-acetoxy-cyclopent-2-enyl ester is re-isolated and-re-subjected to the reaction conditions to generate more acetic acid (1S,4R)-4-hydroxy-cyclopent-2-enyl ester. The process includes:
extracting the aqueous layer with ethyl acetate;
drying the organic layer over MgSO₄, filtering and evaporating;
to the residue Ac₂O (60 g) and Et₃N (60 g) is added directly for an acetylation catalysed with DMAP; and
a distillation of the obtained diacetate (0.2 bar, bp 62° C.) yields to 44.1 g (0.239) light yellow liquid.

The enzymatic hydrolysis reaction was repeated using Novo SP435 as the enzyme, which was found to provide good yields with excellent selectivity and no side reactions, such as for example, further hydrolysis of the monoacetate product to the corresponding diol.

1. A process for the preparation of organic compounds of formula (I): 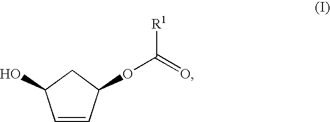
where R¹ is selected from the group consisting of C₁-C₈-alkyl, C₆-C₁₀-aryl, C₁-C₈-alkoxy and C₆-C₁₀-aryloxy, comprising the steps of:
(1) reacting a furfuryl alcohol in an acidic solution for a time sufficient to form a compound of formula (II): 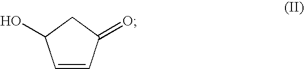
(2) reacting a compound of formula (II) with a protecting group in an aprotic solvent in the presence of base for a time sufficient to form a compound of formula (III): 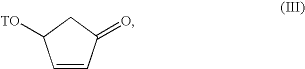
where T is a protecting group;
(3) reducing a compound of formula (III) and removing said protecting group of said compound of formula (III) to provide a compound of formula (IV): 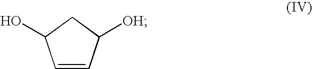
(4) reacting a compound of formula (V): 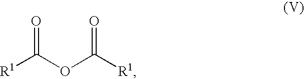
where each R¹ is independently selected from C₁-C₈-alkyl, C₆-C₁₀-aryl, C₁-C₈-alkoxy, and C₆-C₁₀-aryloxy, or
a compound of formula (Va): 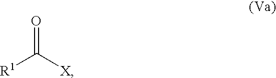
where X is selected from the group consisting of halogen, imidazole or N-hydroxybenzotriazole with a compound of formula (IV) to provide a compound of formula (VI): 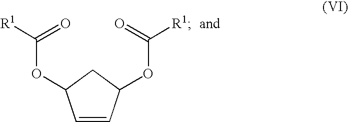
(5) reacting a compound of formula (VI) with an enzyme to provide a compound of formula (I).

2. A process according to claim 1 where the process is for the preparation of organic compounds of formula (Ia) 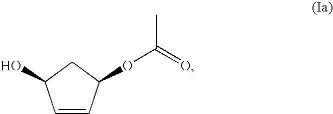
comprising the steps of:
(1) reacting a furfuryl alcohol in an acidic solution comprising water for a time sufficient to form a compound of formula (IIa): 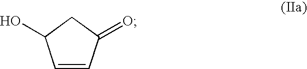
(2) reacting a compound of formula (IIa) with chloro-trimethylsilane in dichloromethane in the presence of base for a time sufficient to form compound of formula (IIIa): 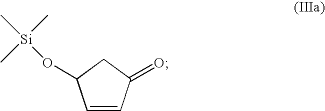
(3) reducing a compound of formula (IIIa) in an aprotic solvent to provide racemic mixture of a compound of formula (IVa): 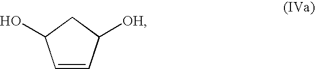
(4) reacting said racemic mixture of a compound of formula (IVa) with acetic anhydride in an aprotic solvent in the presence of base for a time sufficient to form a compound of formula (VIa): 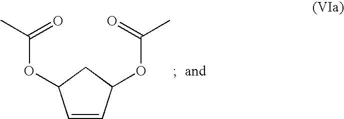
(5) reacting a compound of formula (VIa) with Novo SP435 or Lipase PS Amano to provide a compound of formula (Ia).

3. A process according to claim 2, for the preparation of the compound of formula (Ia), wherein said acidic solution of step (1) comprises ortho phosphoric acid.

4. A process according to claim 2, for the preparation of the compound of formula (Ia), wherein said acidic solution of step (1) has a pH of about 3.0 to about 5.0.

5. A process according to claim 2, for the preparation of the compound of formula (Ia), wherein said base of step (2) is triethylamine.

6. A process according to claim 2, for the preparation of the compound of formula (Ia), wherein step (2) further comprises 4-dimethylaminopyridine as a nucleophilic catalyst.

7. A process according to claim 2, for the preparation of the compound of formula (Ia), wherein step (3) comprises diisobutylaluminium hydride as a reducing agent in step (3).

8. A process according to claim 2, for the preparation of the compound of formula (Ia), wherein said aprotic solvent of step (3) is a mixture of toluene and tert-butyl methyl ether.

9. A process according to claim 2, for the preparation of the compound of formula (Ia), wherein said base of step (4) triethylamine.

10. A process according to claim 2, for the preparation of the compound of formula (Ia), wherein step (4) further comprises 4-dimethylaminopyridine as a nucleophilic catalyst.

11. A process according to claim 2, for the preparation of the compound of formula (Ia), wherein said aprotic solvent in step (4) is dichloromethane.

12. A process according to claim 2, for the preparation of the compound of formula (Ia), wherein step (5) provides an enantiomeric ratio of the product, compound (Ia), of at least 80%.

13. A process according to claim 2, for the preparation of the compound of formula (Ia), wherein step (5) provides an enantiomeric ratio of the product, compound (Ia), of at least 90%.
